(19) United States
(12) Patent Application Publication
(10) Pub. No.: US 2013/0096516 A1
(43) Pub. Date: Apr. 18, 2013

(54) Healing Wounds by Restoring Physiological Norms

(71) Applicant: Joseph H. Schaefer, Grafton, WI (US)

(72) Inventor: Joseph H. Schaefer, Grafton, WI (US)

(21) Appl. No.: 13/692,617

(22) Filed: Dec. 3, 2012

Related U.S. Application Data
(63) Continuation-in-part of application No. 13/299,184, filed on Nov. 17, 2011.
Application No. 13/299,184 is a continuation-in-part of application No. 12/537,755, filed on Aug. 7, 2009.

(51) Int. Cl. A61M 35/00

(52) U.S. Cl. CPC A61M 35/00
USPC 604/290

(57) ABSTRACT

A method of treating a wound by restoring a bodies' physiological norms is disclosed. A wound dressing is applied over the wound in which the wound dressing has an inlet and an outlet and furthermore forms a substantially airtight cover over the wound. A vapor is supplied to the wound dressing via the inlet. The wound is warmed by condensing the vapor into a condensate on a surface of the wound, thereby releasing a latent heat of vaporization and adding moisture to the wound. The excess condensate is removed from the wound dressing via the outlet.

### CROSS-REFERENCE TO RELATED APPLICATIONS

This application is a continuation-in-part application of U.S. non-provisional patent application Ser. No. 13/299,184 entitled “Method of Wound Treatment Using Condensation Warming” filed on Nov. 17, 2011 which is a continuation-in-part application of U.S. non-provisional patent application Ser. No. 12/537,755 entitled “Wound Ventilation System” filed on Aug. 7, 2009. The content of both of those applications are incorporated by reference as if set forth in their entirety herein.

### BACKGROUND OF THE INVENTION

There is a great need for a better way of healing wounds. The present wound healing therapies are not doing an adequate job. There are too many wounds that do not heal and too many resulting complications and amputations.

As some examples, a wound site that becomes hypoxic is known to be at a greater risk of infection, a wound site that becomes hypothermic is thought to be at a greater risk of delayed healing, and a wound with improper moisture can impair healing. When wounds are too dry, they form a scab which slows the healing process. When wounds are too wet, maceration can occur, which also slows healing.

An improved way to heal wounds and avoid complications associated with the multiple conditions listed above is needed.

### SUMMARY OF THE INVENTION

When everything is normal, the human body has a sense of physical ease or comfort associated with good health. A healthy body naturally maintains a closely regulated internal environment conducive to life. This healthy environment includes normal levels of blood flow, temperature, and moisture.

A wound typically disrupts one or more of these levels, placing additional stresses on the body. This disruption starts the healing process when the body is no longer in its healthiest state. The intervention strategy of this invention is to improve health and healing in all wounds by restoring to normal the physiological levels of blood flow, temperature, and moisture in situations where the body is unable to do this itself.

The disclosed device and method of wound treatment uses localized condensation warming in such a way as to restore at least these three physiological norms at the location of the wound simultaneously and continuously.

Near a wound site, normal blood flow decreases due to vascular damage as well as thermoregulatory vasoconstriction. A poor blood supply may result in wound hypoxia as the required oxygen needs to reach the wound via the bloodstream. Hypoxic wound tissue is easily infected and heals poorly. Condensation warming, which is the opposite of evaporative cooling, is provided to the wound bed by this invention. This warming can increase tissue perfusion and oxygenation by preventing local vasoconstriction and increasing the blood flow to the wound edges and surrounding tissues. This may also increase tissue oxygen tension, aid oxidative killing of microbes, and increase resistance to infection. Restoring blood flow to near normal levels with condensation warming has the potential to reduce delayed healing and infection in wounds where thermoregulatory vasoconstriction is occurring.

Normal temperatures are not typically maintained after a wound occurs because of the loss of an insulating layer of skin, resulting evaporative cooling, and less blood flow to bring warmth to the area. Temperature in the human body is both normal and optimal at 37 degrees C. Many wounds are substantially below this optimal temperature with the coldest sometimes being the most difficult to heal. Restoring normal temperature to the wound area with condensation warming has the potential to improve healing by speeding up cellular physiological functions.

Normal moisture levels are disrupted when the normal skin barrier is cut or damaged, exposing the inner tissues to the atmosphere. If the tissue cells dry out, they can lose perfusion and die in the process. Condensation occurring directly on cool wound tissues adds moisture to these wounds. Normal moisture levels may be restored with a wound dressing that mimics the natural moisture barrier originally provided by a healthy skin layer. The active dressing of this invention maintains a moisture balance by adding or removing moisture as needed.

Condensation warming has the potential to prevent hypoxia, hypothermia, and inadequate moisture levels at a wound site. The condensation warming and moisture addition functions of this invention are provided by a heated humidifier similar to those used in respiratory care. The moisture removal function is provided by a connected vacuum source. The heated humidifier and vacuum source work together as follows.

According to one form of the invention, a gas source, along with a liquid water source, enters the inlet of a heated humidifier. The humidifier uses heat energy to both heat the gas and to vaporize the water into the gas. Upon evaporating, the water vapor absorbs the latent heat of vaporization. This gas, now containing water vapor, exits the humidifier outlet at a dew point temperature of 37 degrees C. Next, the saturated gas and vapor enters the inlet of a substantially airtight wound dressing. Inside the dressing, moisture condenses directly on wound tissue cooler than 37 degrees C. The condensate releases its latent heat which gently warms the cool wound tissues. The condensate also adds its moisture to the wound tissues. The gasses, excess condensate, non-condensed vapors, and excess body fluids exit the dressing outlet, through a liquid trap, to a vacuum source.

Sensible heat is heat that changes the temperature of a substance. In comparison, latent heat (sometimes called hidden heat) is heat that is absorbed or released upon a change of state (gas, liquid, solid) of a substance without a change in temperature. The heat content difference between the two can be very significant. For example, the amount of sensible heat released when one pound of water is cooled by one degree Fahrenheit is 1 B.T.U. However, the amount of latent heat released when one pound of water is condensed is 970 B.T.U.s.

This invention primarily uses latent heat of vaporization in lieu of sensible heat. This is done because of the comparably larger quantities of heat that can be transferred at normal body temperature. The use of a dew point temperature which coincides with normal body temperature allows the latent heat to be released to the wound tissues at this optimal temperature. Lower temperatures may not sufficiently prevent vasoconstriction and higher temperatures may cause harm.

Latent heat is absorbed outside a wound dressing so it may be released inside the dressing. The change of state from a liquid to a vapor (evaporation) occurs above 37 degrees C. outside the dressing. The change of state from a vapor to a liquid (condensation) occurs at 37 degrees C. inside the wound dressing. Accordingly, a large amount of heat can be transferred from the outside to the inside of the dressing without the use of potentially harmful elevated temperatures. This heat is transferred by conduction which allows for deeper and quicker warming, as compared to convection. The heat is distributed within the dressing based on demand, with the coldest wound tissues receiving the greatest amount of heat. The amount of heat released is self-regulating and varies inversely with the temperature of the wound tissue.

Moisture is added to the wound bed with the near continuous condensation deposited on wound tissues at or cooler than 37 degrees C.

Moisture is removed from the wound bed when excess liquids are wicked up by the dressing material and continuously aspirated out of the dressing by the vacuum source.

Condensation warming improves wound tissue perfusion and oxygenation by preventing thermoregulatory vasoconstriction at the wound site. Sympathetically induced vasoconstriction may also be stimulated by pain, or blood-volume deficit, which may be treated separately.

Notably, the active wound dressing of this invention uses a singular approach to wound healing. Typical wound dressings are passive, and different designs each provide their own specific levels of permeability, absorptive capacity, and sometimes moisture addition. However, wounds are dynamic and changes in the wound may require changes in the absorption or addition of moisture, which a passive dressing cannot do. It can be difficult at times to properly match the dressing type to the wound. Utilizing the single active wound dressing of this invention eliminates having to choose from multiple dressing types under changing wound conditions. This approach simplifies decision making, eliminating possible errors.

These and still other advantages of the invention will be apparent from the detailed description and drawings. What follows is merely a description of some preferred embodiments of the present invention. To assess the full scope of the invention, the claims should be looked to as these preferred embodiments are not intended to be the only embodiments within the scope of the claims.

### BRIEF DESCRIPTION OF THE DRAWINGS

FIG. 1 illustrates one embodiment of this wound warming system showing how it connects to other wound healing components. The relative size of the wound warming system may be different than shown.

FIG. 2 is the wound warming system of FIG. 1 installed in an enclosure and comprising a wound warming device.

### DETAILED DESCRIPTION OF THE INVENTION

Hereinafter, the wound warming system may be referred to as a “wound incubator”, which is defined as an apparatus used to maintain environmental conditions suitable for the healing of wounds. Incubators are used in tissue culture rooms to grow stem cells, skin fibroblasts and other types of cells. Incubators are used in microbiology for growing bacteria and other microorganisms. This “wound incubator” is meant to mimic the fetal environment and encourage a rapid proliferation of new cells to replace those lost or damaged. A protective environment of controlled temperature and moisture is maintained inside the dressing. The result is an ideal wound environment for cellular growth much as a greenhouse provides the best environment for the growth of healthy plants.

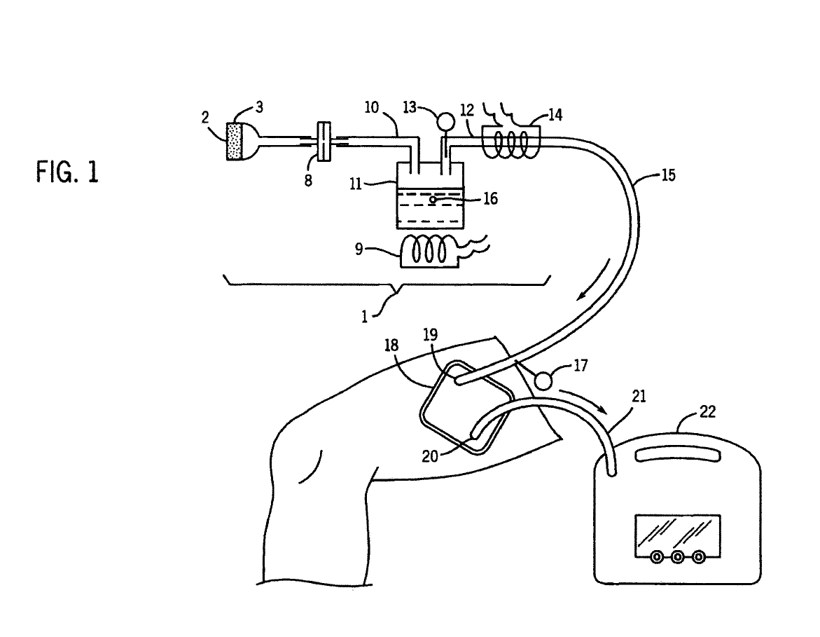

FIG. 1 is a schematic showing the relationship between the wound incubator 1, the wound dressing 18, and the wound vacuum unit 22. This drawing also shows how these components are connected with each other. The flow of gasses through the wound incubator 1 and the wound dressing 18 is powered by the suction force created by the wound vacuum unit 22 or other vacuum source. The direction of the flow of gasses through the various components is illustrated using arrows adjacent the conduits or tubes.

Referring again to FIG. 1, air is always available to be freely drawn in through the air intake 2 after passing through a bacterial filter 3. Flow rates are measured in liters/min. The flow rate is governed by a flow limiting device called the flow rate controller 8. This device may be adjustable, but once a desired total flow rate is known, a constant flow rate makes component design and control simpler. A good constant flow rate device is a filtered orifice flow restrictor. A filter protects the tiny orifice from plugging up. Although not restricted to this range, the flow rate is typically in a range of 0.5 to 1.0 liters/min. This flow rate is preferably high enough to sufficiently warm most wounds and aspirate the dressing liquids; and low enough to be well below the capacity of the vacuum source. The flow rate controller 8 establishes a pressure differential that sets a boundary between atmospheric pressure upstream of the flow rate controller 8 and the negative pressure downstream of the flow rate controller 8 created by the wound vacuum unit 22. When the vacuum unit 22 is run to create the pressure differential, the gas is drawn through the flow rate controller 8 into the humidifier inlet 10.

The purpose of the humidifier 11, its water source 16, its heater 9, and secondary heater 14 is to provide active warming and moisture addition by delivery of gas saturated with water vapor at 37 degrees C. to the wound dressing inlet 19 while minimizing condensation along the way. There are many ways to do this. For ease of explanation, FIG. 1 shows the humidifier 11, the humidifier heater 9 and the secondary heater 14 as being separate components. Most modern heated humidifiers combine such components into a package. Heated humidifiers are also used when supplying respiratory gasses to a patient. They are available in many different designs, including: heated passover humidifier, by-pass humidifier, wick humidifier, vapor transfer cartridge, and capillary force vaporizers. Most would be appropriate for this application, providing they could be downsized for the lower flow rates. The humidifier could be controlled by using sensor 13 and maintaining a 37 degree C. dew point at this location. Preferably, the humidifier produces water vapor only. Unlike mist or droplets, molecules of water vapor are too small to transport bacteria to the wound, which could compromise the wound or create an infection. For the humidifier's water source, distilled water would typically be used and it could be supplied from a refillable container. A sterile IV type bag of water could also be used.

Between the humidifier outlet 12 and the wound dressing inlet 19 is the dressing delivery conduit or tubing 15 which places the humidifier 11 in fluid communication with the wound dressing 18. This delivery tube 15 can be short to reduce heat loss. In any event, there can be some heat loss in this delivery tube 15. If the temperature drops below 37 degrees C. at any location before the dressing inlet 19, there would be unwanted condensation. One way to minimize delivery tube condensation is to add heat with a secondary heater 14. This secondary heat source adds sensible heat to offset the loss of sensible heat in the delivery conduits as the vapor travels away from the humidifier 11. This added heat would slightly exceed the heat loss and could be controlled by using sensor 17 located as close as possible or within dressing inlet 19. In some forms, the secondary heater 14 can be a heating element or sleeve around some or all of the tube 15 between the humidifier 11 and the wound dressing 18 to try to maintain the temperature of the vapor at just above 37 degrees C. prior to the inlet 19. In lieu of secondary heater 14, condensation could also be minimized by using a by-pass humidifier, a heated delivery tube, an insulated delivery tube, or a heated wire breathing circuit.

There are many methods of controlling the chosen humidifier and associated heat sources so that heated gasses enter the wound dressing 18 slightly warmer than their 37 degrees C. dew point temperature. The wound dressing 18 may be chosen from among the many available existing negative pressure dressings and is designed to be substantially airtight. Most likely, an inlet tube connection 19 can be added to the typical dressing. This connection is preferably added in a location non-adjacent to the existing outlet tube connection 20.

The wound is then warmed and moisture is added by condensing the vapor in the saturated gas into a condensate on a surface of the wound, such as the wound tissues, within the dressing 18. This condensation of the vapor onto the wound releases the latent heat of vaporization, thereby locally warming the area of the wound and the surrounding tissues. Moisture is added to the wound bed with the near continuous condensation deposited on wound tissues at or cooler than 37 degrees C. Before evaporation threatens to dry tissues, evaporative cooling would lower the tissue temperature to 37 degrees C., causing the addition of more condensation and associated warmth. Because of this, the dressing environment is expected to remain fully saturated and at 37 degrees C.

Too much moisture or exudate at the wound site can cause maceration of the wound tissues and interrupt healing. Moisture can be removed from the wound bed when excess liquids are wicked up by a dressing material. As the liquids accumulate within the dressing, they can be continuously aspirated out of the dressing by the vacuum source (e.g., vacuum unit 22). Moisture is added and removed, as needed, to maintain a continuous moisture balance.

Autolytic debridement can liquify unwanted necrotic burden in a moist wound without harming healthy tissue. Continuous moisture balance makes continuous autolytic debridement possible. The accumulation of necrotic burden is managed with this maintenance phase of debridement.

Between dressing changes, when the wound is exposed, it is common practice to cleanse a wound. Wounds are cleansed to aid the removal of exudates, debris, slough and to prevent infections. The flow of condensate through this active wound dressing can also perform a cleansing function which is continuous and not relegated to just between dressing changes. The flowing condensate also dilutes the wound exudate, prevents thickening, and allows the vacuum source to remove more exudate.

One problem encountered during dressing changes is the adherence of wound tissue to the dressing material. Forcibly removing the dressing can cause trauma and disrupt the healing process. With this active wound dressing, the condensate created on the wound surface can soak and help soften the bonds between the tissue and dressing, minimizing the occurrence of this problem.

Condensation may form on the inside of the outer surface of the dressing 18 when its temperature is below 37 degrees C. To minimize this condensation, the dressing 18 may be covered with an insulated pad or blanket. This will reduce the heat loss and keep the dressing 18 warmer.

With respect to the vacuum source in the illustrated embodiment, dressing exit tubing 21 extends to the remotely-located wound vacuum unit 22. This wound vacuum unit 22 may be chosen from the many types available. The operation of this unit is to be as per the manufacturer's instructions. This added “wound incubator” is not meant to change any of the operational or safety requirements of the typical negative pressure wound pump as recommended by the vacuum unit manufacturers. However, a wound vacuum unit is not necessary. Instead, another vacuum source, such as a wall outlet vacuum, may also be used along with a suction unit, regulating the vacuum and collecting the liquids. The vacuum source may also be the power source that creates flow or wound ventilation. This can eliminate the need for a fan or the necessity for pressure induced flow.

A wound site that experiences any degree of vasoconstriction is not receiving the optimal perfusion and oxygenation needed to provide a good environment for healing. By controlling dew point and condensation, both the temperature and moisture at the wound site can be advantageously regulated so as to restore the body's normal physiological levels including blood flow, temperature and moisture. The wounds having the greatest deviations from normal would be expected to benefit the most. The restoration of physiological norms may begin the healing process.

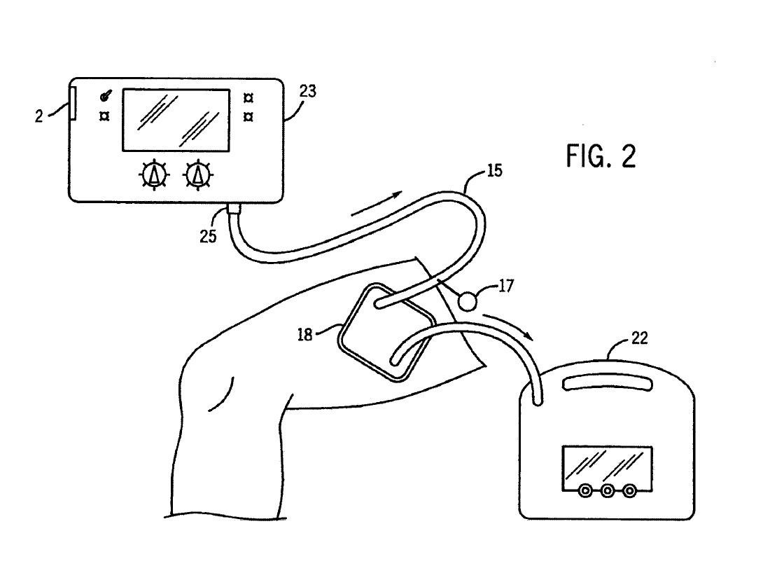

While FIG. 1 is used to show basic components and principles of operation, FIG. 2 is used to show these same components (i.e., the components that are part of the wound incubator 1) enclosed in a wound incubation device 23. This wound incubation device 23 contains a filtered air intake 2 and an outlet connection 25 for connection to the wound dressing 18 via the delivery tubing 15. The face of this device 23 may be used to mount operational controls and display desired information.

Because of the fear of possible contamination, all components touched by the water are typically designed for single patient use like those used in respiratory humidifiers. The wound incubator may be configured to be mounted on an IV pole or on a bed rail, placed on a bedside table or designed to be portable. It should be located close to the wound site. It uses an electrical power source from a battery or a 120V power cord. Temperature sensor 17 communicates with the wound incubation device 23. The wound vacuum unit 22 may also be configured to communicate with the wound incubator.

Another embodiment would have the components of the wound incubator and the components of the vacuum source contained in a single enclosure. Both of these devices would not only share a single enclosure, they also would share a power supply, electronics, displays and communications. A single enclosure would save space, costs, and be simpler to hook up and operate. Dressing inlet and outlet tubes would go to the same device.

This wound warming method could be suitable for cooler than normal wounds and those where thermoregulatory vasoconstriction may be occurring. These wounds would include those on humans as well as warm-blooded animals. With animals, the dew point temperature used would correspond to that particular animal's normal body temperature.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

1. A method of treating a wound by restoring a bodies' physiological norms, the method comprising:
applying a wound dressing over the wound, the wound dressing having an inlet and an outlet and forming a substantially airtight cover over the wound;
supplying a vapor to the wound dressing via the inlet;
condensing the vapor into a condensate on the surface of the wound to both:
release latent heat to warm the wound,
release liquid to moisturize the wound, and
removing an excess amount of the condensate and an uncondensed portion of the vapor from the wound dressing via the outlet.

2. The method of claim 1 further comprising preventing local thermoregulatory vasoconstriction by warming the wound.

3. The method of claim 1 further comprising speeding up a rate of cellular physiological functions by warming the wound.

4. The method of claim 1 further comprising maintaining a desired moisture balance at the wound by regulating the condensate used to moisturize the wound and the excess amount of the condensate removed via the outlet.

5. The method of claim 1 wherein the method simultaneously prevents hypoxia, hypothermia and inadequate moisture levels at a site of the wound.

6. The method of claim 1 wherein a dew point temperature of the vapor coincides with normal human body temperature (37 degrees C.), thereby allowing the latent heat to be released to tissues of the wound at this temperature without elevating a temperature at the vapor.

7. The method of claim 1 wherein heat transferred within the dressing is based on a temperature of tissues of the wound with the tissues of the wound that are coldest receiving a greatest amount of heat in a self-regulating manner.

8. The method of claim 1 further comprising transferring heat from the condensate to tissue of the wound by conduction which allows for deeper and quicker warming, as compared to convection.

9. The method of claim 1 further comprising absorbing latent heat outside a wound dressing and releasing at least a portion of the latent heat is inside the wound dressing.

10. The method of claim 1 wherein the condensate is water.

11. The method of claim 1 further comprising a step of evaporating a liquid from a liquid source to form the vapor prior to supplying the vapor to the wound dressing via the inlet, in which the step of evaporating includes absorbing the latent heat of vaporization into the vapor.

12. The method of claim 11 wherein the step of evaporating the liquid from the liquid source is performed by a humidifier that heats and humidifies the liquid to form the vapor.

13. The method of claim 1 wherein a vacuum source in fluid communication with the outlet performs the step of removing the condensate and the uncondensed portion of the vapor from the wound dressing via the outlet.

14. The method of claim 13 further comprising the step of drawing the vapor through the wound dressing using the vacuum source exclusively, thereby eliminating a need for a positive pressure source.

15. The method of claim 1 further comprising controlling a flow of a gas source to the inlet using a gas flow controller.
